(19) United States
(12) Patent Application Publication
(10) Pub. No.: US 2026/0188437 A1
(43) Pub. Date: Jul. 2, 2026

(54) SYSTEM FOR ONLINE PROCESS, CHEMOMETRIC ANALYSIS, USING MACHINE-LEARNING

(71) Applicant: Modcon Systems Ltd., London (GB)

(72) Inventors: Gregory Shahnovsky, Haifa (IL); Ariel Kigel, Haifa (IL); Gadi Briskman, Haifa (IL); Tom Rosenwasser, Haifa (IL)

(73) Assignee: Modcon Systems Ltd., London (GB)

(21) Appl. No.: 19/004,593

(22) Filed: Dec. 30, 2024

(51) Int. Cl. G16C 20/70; G06N 20/00

(52) U.S. Cl. CPC G16C 20/70; G06N 20/00

(57) ABSTRACT

The invention is a system and method for using spectroscopy and precision machine-learning models for accurate chemometric analysis of online process constituents.

### TECHNICAL FIELD

The invention is a system for chemical analysis of online process constituents using spectroscopy coupled with machine-learning modeling.

### BACKGROUND OF INVENTION

The crux of refining processes is effective process control, which ensures operations are executed with peak efficiency, unerring accuracy, and minimized costs.

Incorporating online process analyzers is universally recognized as a crucial step to prevent financial losses, often resulting from redundant re-processing or superfluous giveaways.

Needs assessment have gone beyond just the addition of online process analyzers to include the interdisciplinary field of chemometrics, which fuses mathematical and statistical methodologies to extract valuable information from chemical datasets. Essentially, chemometrics employs mathematical models to unravel complex chemical datasets, illuminating hidden patterns or relationships. In the industrial context, the vastness and complexity of chemical data necessitate tools like chemometrics to distill actionable insights.

Some software platforms have been pivotal in facilitating chemometric analyses. While powerful, these platforms often demand a robust understanding of both the intricacies of chemometrics and the software mechanics—a steep learning curve that can deter many from adopting them. Nevertheless, the benefits of merging machine-learning into chemometrics include efficiency, scalability and adaptability so long as the solution reduces the need for robust understanding of chemometrics.

### BRIEF DESCRIPTION OF THE INVENTION

The invention herein disclosed comprises a spectroscopic subsystem coupled to a machine-learning module (MLM) wherein the labeled data being used to train the MLM incorporates expert understanding of chemometrics, provided by an automated spectral processing subsystem, such that ultimately a ML model is built that is specific to the online process and can provide near real-time sample analyses while operated by persons who need not have any chemometric background.

As with any ML-based system, the model will ultimately be based on training and refinement over the course of a large number of samples. In addition, the model's accuracy is in large part dependent upon weeding out outlier samples from a large number of samples. The model must also be dynamic such that as baselines change with changes in process constituents, the model can adapt to those changes and continue to provide accurate analyses.

Initially, as spectral results for samples are collected, analyses and the resulting boundaries are used to define outlier determination and elimination. As different processes are put in place, or different baselines emerge, the ML module and resulting ML model must be refined, accordingly, so that the model is inextricably linked to the online process for which it is used.

NIR spectroscopy may be used because the interaction between NIR light impinging on sampled flow instances involves molecular rather than sub-molecular spectral results. This can lend itself to faster, more accurate chemical analyses.

The spectroscopy subsystem may be general purpose in that it could be used for a variety of chemicals and processes. The MLM becomes personalized to the specific online process as it is trained with expert-analyzed labeled data. And, the resulting ML model is very specific to the online process and its current process flows.

The precision of the spectrometric measurement of a chemical or material's physical property is only as good as the model used to reveal it. The invention enables an automated way to build spectrometric models. It is based on the concept that the property corresponding to each spectrum can be predicted using the model trained using the rest of the samples in the set with a bounded error. In essence, the calibration set is diverse yet belongs to a reasonably confined distribution of spectra. This is termed “homogeneity.”

To that end, a novel method for removing outliers from the training data is based on homogeneity wherein an “early stopping” criteria is met. The method associated with this concept is adept at establishing models that provide reliably accurate results.

In addition, a novel method for selecting hyperparameter sets is used to quickly find the hyperparameters best suited to model accuracy. Hyperparameters are configuration settings that are specified before the learning process begins in machine learning models, including those used in regression analysis. They play a crucial role in determining how a model learns from data and ultimately performs.

### BRIEF DESCRIPTION OF DRAWINGS

FIG. 1 depicts an embodiment of the spectroscopic subsystem.

FIG. 2 depicts an embodiment of the subsystem in FIG. 1 whose detector is providing spectral results.

FIG. 3 depicts a case wherein spectral results samples may be chemometrically analyzed by an expert to show the conversion from spectral to chemical analysis.

FIG. 4 depicts an embodiment wherein an automated spectral-processing subsystem provides spectral sample data used to train an MLM which, in turn, yields an ML model that is specific to the training data properties.

FIG. 5 shows an embodiment wherein the ML model can receive spectral samples and produce chemical analysis.

FIG. 6 depicts an embodiment wherein using regression analysis outlier boundaries can be determined and used to identify outliers before they are used to train an ML module.

FIG. 7 shows a graph of error versus iteration number as outliers are removed.

FIG. 8 is an embodiment of a method for quickly eliminating outliers until “early stopping” criteria are met.

FIG. 9 is an embodiment of a method for selecting model hyperparameter sets.

### DETAILED DESCRIPTION OF INVENTION

The invention is a system comprising a spectroscopy subsystem, an automated spectral-processing subsystem, a machine-learning module subsystem, and a machine-learning model subsystem. It is operative to support chemometric analysis of online process samples without relying on experts having robust knowledge of chemometrics and mathematical analysis.

Chemometrics is a multidisciplinary field that combines statistical, mathematical, and computational methods to extract meaningful information from large and complex chemical datasets. It begins with the collection of data from various analytical techniques such as spectroscopy and electrochemical methods. After collecting the data, it is essential to preprocess it to ensure quality and consistency. This step typically involves cleaning the data, removing noise, and normalizing or transforming the data to make it suitable for analysis.

Chemometrics employs multivariate analysis techniques to handle the complexity of the data. Unlike classical methods that examine one factor at a time, chemometrics considers all variables simultaneously.

Calibration is a critical step in chemometrics where a model is developed to relate the measured properties of a chemical system to the properties of interest. This involves using a calibration or training data set that includes reference values for the properties to be predicted. The model is then optimized to predict these properties accurately

After developing the model, it is validated to ensure its performance and reliability. Validation involves testing the model with new, independent datasets to evaluate its predictive capabilities. Techniques such as cross-validation, bootstrap, and permutation are used to assess the model's performance and estimate figures of merit like root-mean-squared error and selectivity.

Once the model is validated, it can be used to make predictions on new, unknown samples. Some of the most useful aspects of chemometrics are: identifying underlying patterns and relationships in the data that may not be apparent through traditional analysis methods; developing models that can predict properties or behaviors of chemical systems based on measured data; and techniques to minimize noise and improve the quality of the analytical signal.

Near-Infrared Spectroscopy (NIR) offers several advantages over other spectroscopy alternatives, particularly when supporting chemometric analysis. Some key advantages are: NIR spectroscopy is notably fast, with measurement times ranging from 10 to 60 seconds, allowing for high sample throughput and real-time analysis in process monitoring; and NIR spectroscopy is non-destructive and non-invasive, enabling the analysis of samples without altering or damaging them. In addition, unlike many other spectroscopic techniques, NIR spectroscopy does not require sample preparation. Solids and liquids can be analyzed in their pure form, eliminating the need for chemicals, solvents, or other reagents. NIR spectroscopy is relatively low-cost compared to other methods. It does not generate waste, does not require chemicals or solvents, and the instruments themselves are often less expensive to maintain; and, it can measure both chemical and physical parameters, such as moisture content, API content, hydroxyl value, and viscosity. What's more, NIR light penetrates deeper into the material than other forms of spectroscopy, making it ideal for analyzing heterogeneous samples and providing a more representative result by measuring beyond the surface.

NIR spectroscopy, when combined with chemometric tools, is highly effective for quantitative and qualitative analysis. Chemometric methods such as Principal Component Analysis (PCA), Partial Least Squares Discriminant Analysis (PLS-DA), and Partial Least Squares Regression (PLSR) are commonly used to extract useful information from NIR spectra.

The following figures and descriptions are exemplary and should not be read as limiting the invention scope.

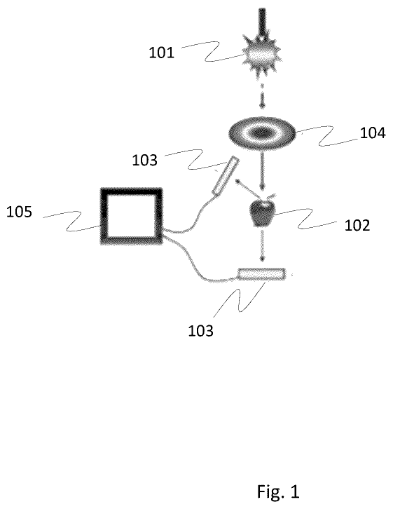

FIG. 1 shows an embodiment of a spectroscopy subsystem. A light source (101) sends light through a filter (104) that passes a particular range of protons and blocks other photons. These post-filtered beams impinge on a sample (102) and interact with its molecules. Depending upon the molecules the light interacts with, some frequencies will be partially absorbed creating a spectral result with peaks and valleys. Detectors (103) convert the reflected or transmitted light into signals which are fed to a computing subsystem (105) and displayed as spectral results showing intensities versus frequencies.

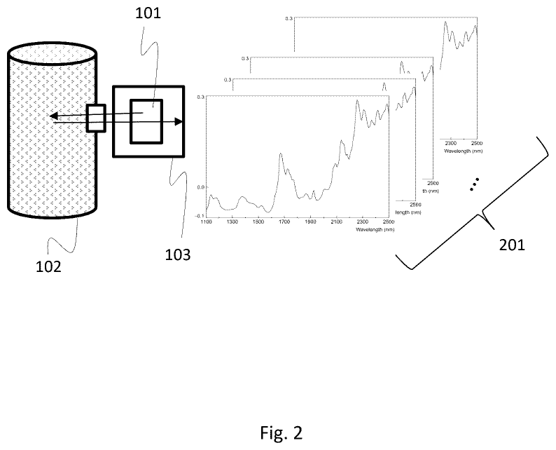

In FIG. 2, the online process samples (102) are impinged by the light source (101) and reflected light, after interacting with molecules in sample 102 are detected by the detector (103) and signals conveyed to a computing subsystem (not shown). The computing subsystem then creates spectral graphics (201) for each sample.

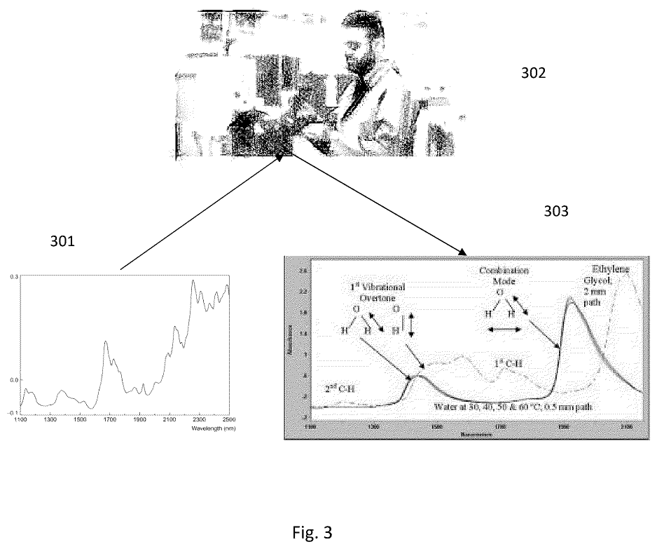

In FIG. 3, selected samples (301) are analyzed by a chemometric-knowledgeable person (302) and the spectral sample results are interpreted as to chemical makeup (303).

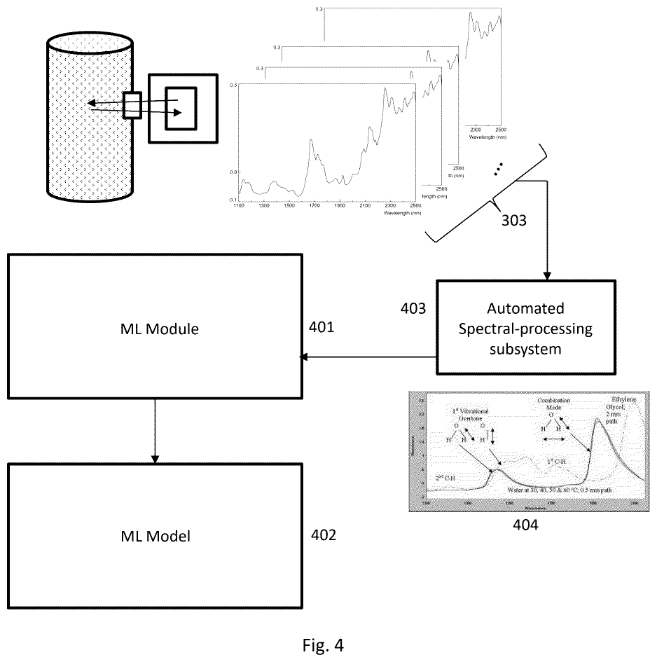

In FIG. 4, an automated spectral-processing subsystem (403) provides interpreted spectral data (404) to an MLM (401) which ultimately creates an ML model (402). Here, the model building is triggered automatically as additional lab results are added to the sample pool. The automated spectral-processing subsystem comprises a data-interface containing the lab results and corresponding spectral files. The lab data is conveyed by the spectroscopy subsystem, to the automated spectral-processing subsystem, and is then conveyed to the ML module without human intervention.

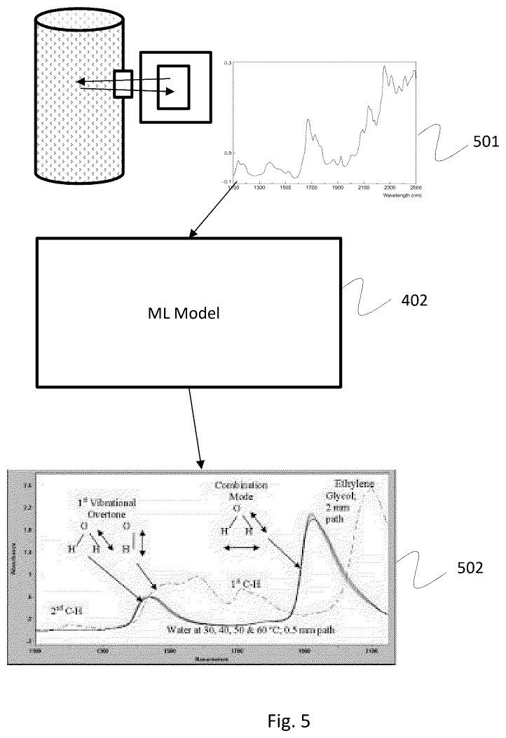

As shown in FIG. 5, once an ML model is created and validated (402) then currently sampled spectral data (501) can be interpreted by the model (402) to produce analyzed results (502) based on the MLM's chemometric training.

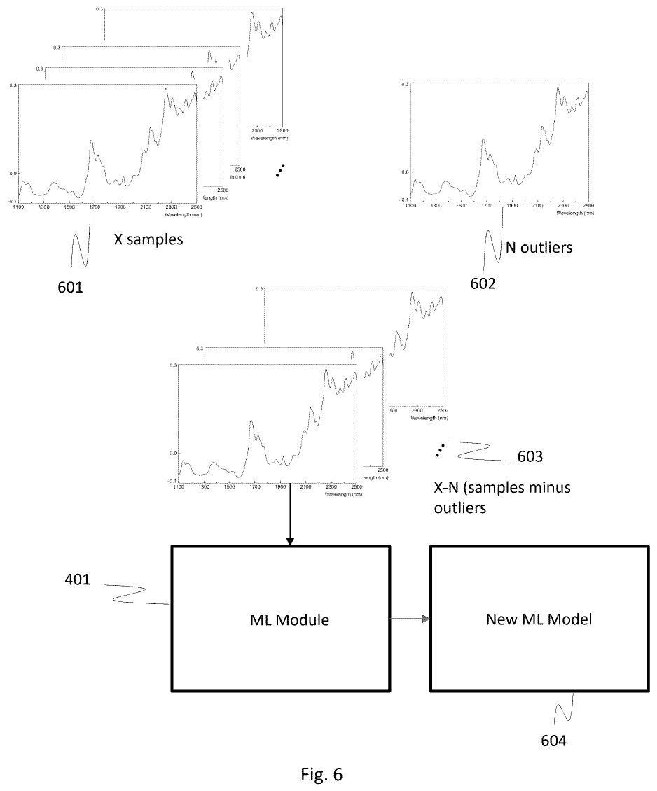

As shown in FIG. 6, the ML model is dynamically modified to create a new ML model (604) essentially adapting to changes in process samples. After X samples (601) have been obtained, N outliers are identified and removed (602). Outliers in regression analysis are identified through a combination of graphical inspection, numerical calculations, and statistical measures, such that these points are carefully examined to determine their impact on the model. The remaining data from the samples X-N (603) are then input to the MLM (401) and may modify the model (604) such that it is relevant to the current state of process constituent properties.

Much of the novelty of the system and method has to do with how the models are established and how outliers are dealt with. There are a variety of ways for performing regression analysis on sample data, for example. In the process, some samples are considered outliers and others are considered part of expected results. To do it right, at first, looks a bit like a chicken-and-egg dilemma. Ultimately, the hyperparameters used for model training are based on regression analysis of samples to remove outliers and reach a point of homogeneity. But, if those hyperparameters are not optimal, the training and resulting model will not be optimal.

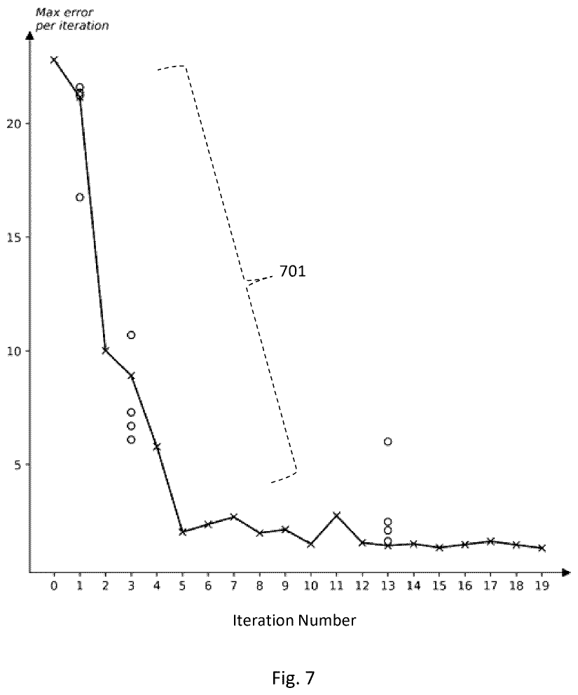

In FIG. 7, the errors clearly diminish as outliers are removed (701). A weakness of partial-least-square (PLS) for spectrometric models is its susceptibility to a low number of outliers in the data used for model calibration. The sample in the training data can be an outlier for several reasons. For example, it may be the wrong lab results. It may be a noisy spectrum that corresponds to the lab result. Or, it may be a skew between the spectrum's timestamp and the time when the sample was taken. The tools typically used to sort out the dilemma are intended for professionals who understand the mathematics that underlie the PLS model. The tools, themselves, make non-trivial decisions about which samples to include and which to discard. Practice has shown that this is a suboptimal approach to outlier detection. For example, sample spectra close to the distribution center may turn out to be outliers whereas a spectrum at the edge of distribution may be valid and should be incorporated into the model.

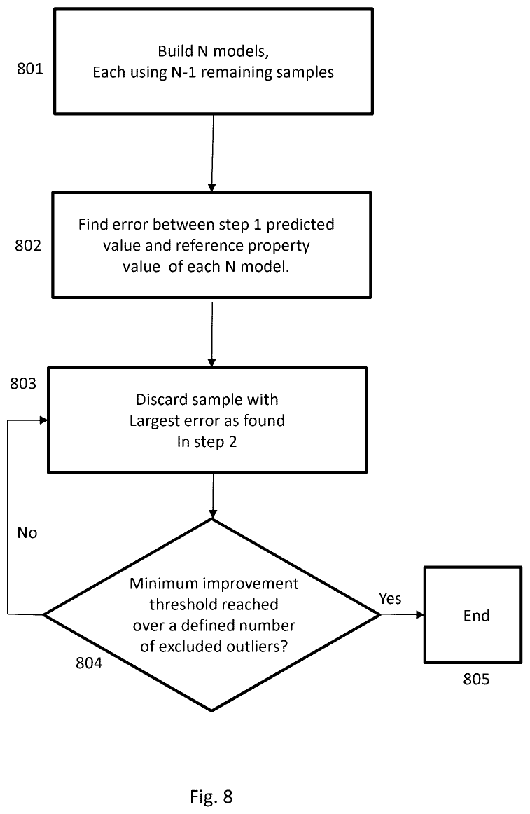

As shown in FIG. 8, a method is used for removing outliers from training data based on homogeneity, that is, when a minimum improvement threshold over a defined number of excluded outliers has been reached. The eliminating cross-validation process comprises building N models, each using N-1 remaining samples (801), then, finding the error between the reference property value that corresponds to each N spectrum file and the prediction of this property per the first step (802). Then, discarding the sample with the largest error as found in the second step (803) and repeating until early stopping criteria has been met (804).

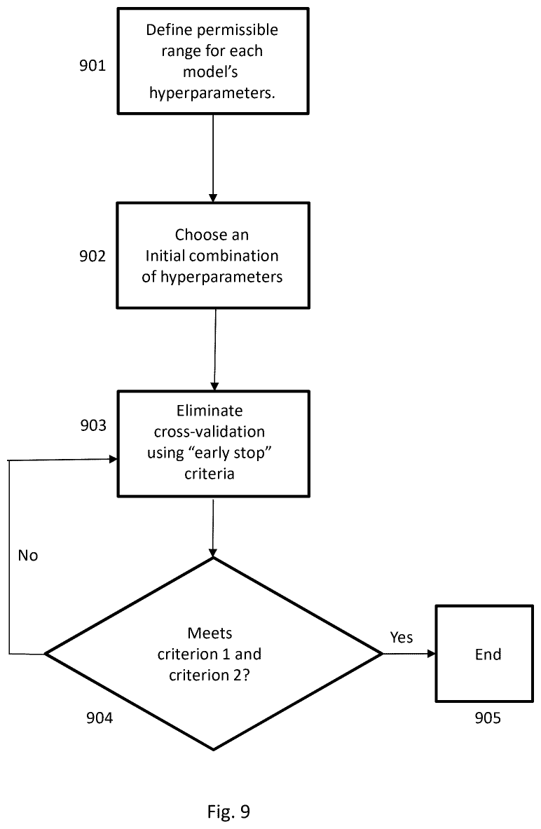

With regard to training and selecting hyperparameters, FIG. 9 shows an embodiment of a method whereby the first step is defining the permissible range of each model's hyperparameter values (901). Then, choosing an initial combination of hyperparameter values (902). Next, executing eliminating cross-validation using early-stopping criteria (903). Repeating this third step for all combinations of hyperparameters (904). And, choosing the set of hyperparameters (905) that satisfies two conditions: 1) the error at the iteration of the early stopping is within a predefined proximity among error values of the evaluated sets of model hyperparameters; and

2) among the sets of hyperparameters that meet condition 1, a set is chosen where the early-stopping criterions was met within the minimal number of eliminated samples.

For further elucidation of the process in FIG. 9, various combinations of hyperparameters are evaluated to find the one that allows excluding a minimal number of samples from a training set. For some models, such as those hosted on neural networks, evaluating every possible combination of hyperparameters may be impractical because it will take too much time. Statistical sampling of hyperparameter combinations can address that problem. Initially, random hyperparameter combinations are evaluated. Then, the more promising areas of hyperparameter space are sampled more frequently. This is known as Bayesian Optimization. The same hyperparameter search process can be repeated after the spectrometric model is built thereby using the cleanest calibration set.

What is claimed is:

1. A system for online process, chemometric analysis comprising:
a spectroscopy subsystem;
an automated spectral-processing subsystem;
a processing subsystem comprising
a computing subsystem comprising:
a central-processing subsystem;
a data-memory subsystem;
a program-memory subsystem;
an input-output subsystem;
at least one system-control program;
a machine-learning module subsystem; and
at least one machine-learning model.

2. A system as in claim 1 wherein:
the spectroscopy system is operative to provide near-infrared spectral imaging.

3. A system as in claim 1 wherein:
the spectroscopy system is operative to provide ultraviolet spectral imaging.

4. A system as in claim 1 wherein:
the automated spectral-processing subsystem is operative to receive lab data input from the spectroscopy subsystem, produce corresponding spectral imaging, interpret the spectral images, and convey the interpreted spectral images to the machine-learning module subsystem.

5. A method comprising:
building a predetermined number, N, of models wherein each model is based on N-1 remaining samples;
finding, for each of N models, error between predicted value and reference property value;
discarding a sample with largest error; and
determining when a minimum improvement threshold has been reached for a defined number of excluded outliers.

6. A method comprising:
defining a permissible range for a machine-learning model's hyperparameters:
choosing an initial combination of the machine-learning model's hyperparameters;
initiating an eliminate cross-validation step using early-stop criteria;
a) identifying hyperparameter sets wherein the error at the iteration of an early stopping is within a predefined proximity among error values of evaluated sets of model hyperparameters;
b) identifying the hyperparameter sets wherein the early-stopping criterion was met within the minimal number of eliminated samples; and
selecting the hyperparameter set that meets the criteria of steps a and b.
